(19) United States
(12) Patent Application Publication
(10) Pub. No.: US 2016/0000508 A1
(43) Pub. Date: Jan. 7, 2016

(54) BARRIER KiT FOR EMT MONITORING EQUIPMENT

(71) Applicant: Darren Finn, Ronkonkoma, NY (US)

(72) Inventor: Darren Finn, Ronkonkoma, NY (US)

(21) Appl. No.: 14/323,315

(22) Filed: Jul. 3, 2014

(51) Int. Cl. A61B 19/02

(52) U.S. Cl. CPC A61B 19/026; A61B 2019/0268; A61B 2019/0201

(57) ABSTRACT

A barrier kit for emergency medical technician sterile patient monitoring equipment provides an emergency medical technician (EMT) a variety of sanitary cover barriers to provide adherence to a cleaning regimen, while avoiding patient to patient cross contamination. The sterile kit is contained in a single envelope including a set of sanitary barriers or sheaths of the same material mounted on a cardboard substrate for a stethoscope, a pulse oximeter probe, a necktie protector and an arm wrap cover to prevent a blood pressure monitor cuff from contacting the patient skin with an attached cover for protection against splatter of bodily fluids from one patient to an EMT technician or from one patient to another patient.

### FIELD OF THE INVENTION

The present invention relates to disposable sanitary covers for patient monitoring equipment in emergency rescue and other medical environments.

### BACKGROUND OF THE INVENTION

It is well known that many patients are affected by hospital acquired infections even after a short stay. Consider that an ambulance is a “rolling hospital” that is used as a short-term “residence” for many patients while transporting them from accidents or emergencies with high potential for contamination from a previous patient to the next. Besides the typical dangers of ordinary bacterial agents such as E-coli, exposure to antibiotic-resistant strains and newer viral and bacterial strains previously unknown in our region such as E-bola, MRSA, and MERS is far more serious. To minimize these risks, the South Western Ambulance Service has put together an Emergency Ambulance Cleaning Schedule that specifies cleaning pulse oximeter probes, stethoscopes, and blood pressure (BP) cuffs after each patient use with detergent wipes. A further directive is that equipment must be disposed of as contaminated waste if Velcro becomes contaminated with body fluids.

The prior art reveals attempts to limit cross contamination between users of medical equipment by providing disposable barriers or sheaths that preclude direct contact of diagnostic equipment to patient skin. Disposable covers for stethoscope diaphragms and heads are the subject of several patents. Exemplary are the patents of Hasbrouck U.S. Pat. No. 3,255,841 for a bell cover, Turner U.S. Pat. No. 4,871,046 for a head shield, Plourde U.S. Pat. No. 4,461,368 for a diaphragm cover, and Gross U.S. Pat. No. 8,387,745 for an anti-bacterial cover.

Also in the prior art are a blood pressure cuff cover of Napolitano et al. (U.S. Pat. No. 5,651,368) and a disposable cover/liner for a BP device of Masciorotte (U.S. Pat. No. 4,967,758). While the Napolitano invention is a sturdy flexible enclosure for the entire cuff, it may be almost as costly as a “disposable” BP cuff which has not been widely accepted as a one-patient use device. The Masciorette patent describes a liner which does preclude contact of a BP cuff with patient skin, but it does not adequately protect the cuff from extraneous bodily fluids and blood splatter.

### OBJECTS OF THE INVENTION

It is therefore an object of the present invention to provide disposable sanitary covers for patient monitoring equipment in emergency rescue and other medical environments.

Another objective of this invention is to provide a kit of disposable barriers/sheaths that are quick and easy to use, offer real contamination protection, and are affordable enough to be used consistently.

Other objects will become apparent from the following description of the present invention.

### SUMMARY OF THE INVENTION

In keeping with these objects and others which may become apparent, the present invention is a kit providing an emergency medical technician (EMT) or other health care provider with a variety of barriers to either augment, eliminate, or at least simplify adherence to the cleaning schedule directive. It even addresses the case of the EMT or other health care provider to patient cross contamination. The sterile kit is contained in a single envelope approximately 14″ by 19″ by ⅛″ thick (36×48×0.3 cm). It contains a family of barriers or sheaths of the same material mounted on a cardboard substrate for a stethoscope (including head), a pulse oximeter probe, and, preferably, an optional necktie protector (which also doubles as a disposable bib). In addition, separately inside the same envelope is an arm wrap to prevent the BP cuff from contacting the patient skin with an attached cover which is draped atop the BP cuff for protection against splatter of bodily fluids.

Therefore, in general, a barrier kit for EMT monitoring equipment includes a sterile envelope of disposable barrier material. The envelope contains a board of disposable sterile material on which are mounted a family of sanitary barriers and sheaths. The board has a releasable adhesive on one surface thereof on which the barriers and sheaths are releasably mounted. The family of barriers and sheaths a kit including preferably a finger sheath adapted for use over a patient finger for isolating contact with a pulse oximeter or pulse oximeter probe, a barrier cover for a stethoscope, an optional single layer sheet with fold lines adapted to enclose a tie or chest region of an emergency medical personnel person, and a barrier sheet adapted for wrapping around an upper arm of a patient to provide a smooth base surface for a blood pressure cuff. The aforementioned barriers and sheaths are made from materials resistant to migration of microbes therethrough.

Preferably the barrier cover for a stethoscope comprises a long section for enclosing a sound hose of the stethoscope, a stethoscope head at a proximal end thereof, and tubing forming ear pipes at an open Y-shaped section at a distal end thereof, a long section being foldable for mounting on the board so that the board fits within the sterile envelope. The Y-shaped section is provided with a pair of fasteners, such as, for example, adhesive tabs for attaching the Y-shaped section around the tubing of the stethoscope.

In an optional embodiment, the barrier cover for the stethoscope includes only a head cover with a flat bottom surface for mounting on the board, wherein the head cover has an access slit on a top surface thereof, the head cover being round to accommodate and enclose the generally circular head portion of the stethoscope.

With respect to the optional single layer sheet for either enclosing the tie of an emergency medical personnel person or for acting as a bib on the chest region of that person, the single layer sheet preferably has a pair of spaced, parallel fold lines for enclosing the tie, and the single layer barrier sheet is retained on the board by a plurality of fasteners, preferably a plurality of releasable adhesive patches. Preferably, the kit includes an optional tie pin for securing the single layer sheet enclosing the tie to a shirt of an emergency medical personnel person or other health care provider. Further, optionally the single layer sheet is adaptable for use as a bib when fully unfolded and attached to a shirt of the EMT person or other health care provider attending to the patient.

Preferably the separate barrier sheet for covering a blood pressure cuff includes narrower and wider bands of stretch wrap, where the wider band provides a smooth base surface for installation of the blood pressure cuff, and the narrower band being thinner, where the wider band preferably has a low tack surface on a side facing the arm of the patient.

In use, a method is provided for using disposable sanitary covers for patient monitoring equipment in emergency rescue and other medical environments comprising the steps of:

a) providing sterile envelopes of disposable barrier material in an EMT emergency vehicle;

b) inserting into each respective envelope a board of disposable sterile material on which are mounted a family of sanitary barriers and sheaths, the board having a releasable adhesive on one surface thereof on which the barriers and sheaths are releasably mounted, the family of barriers and sheaths comprising a sheath adapted for use over a patient finger for isolating contact with a pulse oximeter or pulse oximeter probe, a barrier cover for a stethoscope, and an optional single layer sheet with fold lines adapted to enclose a tie or chest region of an emergency medical personnel person, the barriers and sheaths being made from materials resistant to migration of microbes therethrough;

c) separately enclosed in the sterile envelope, a barrier sheet adapted for wrapping around an upper arm of a patient to provide a smooth base surface for a blood pressure cuff; the sheet being made from material resistant to migration of microbes therethrough;

d) carrying the envelopes containing the families of sanitary barriers and sheaths in the emergency vehicle carrying medical technicians when traveling to a site having a medical emergency;

e) in an emergency situation dealing with a victim patient, opening one of the envelopes and withdrawing the board within with the family mounted thereon;

f) removing the finger sheath from the board and sliding the finger sheath onto a distal end of a finger of the patient in preparation for measuring oxygen levels in blood of the patient;

g) removing the barrier cover and enclosing the stethoscope in the barrier cover prior to deploying the stethoscope;

h) optionally using the single layer sheet to enclose a tie of an emergency medical personnel person if the emergency medical personnel person is wearing a tie or deploying the single layer sheet as a bib on a medical technician;

i) wrapping the barrier sheet around the upper arm of the patient with said narrower band being closest to the arm pit of the patient, followed by placing said blood pressure cuff around the wider band of the barrier sheet and then pulling the narrower band down covering the blood pressure cuff; and

j) the respective barriers and sheaths being made from materials resistant to migration of microbes therethrough, whereby cross contamination between successive patients is reduced.

### BRIEF DESCRIPTION OF THE DRAWINGS

The present invention can best be understood in connection with the accompanying drawings. It is noted that the invention is not limited to the precise embodiments shown in drawings, in which:

FIG. 1 is a front elevation of the sterile EMT barrier kit of this invention.

FIG. 2 is a plan view of the family of barriers/sheaths formed of the same plastic film and mounted on a disposable cardboard substrate.

FIG. 3 is a perspective view of the procedure of donning a pulse oximeter sheath prior to using a pulse oximeter.

FIG. 4 is a plan view showing a stethoscope inside the sheath of this invention.

FIG. 5 is a perspective view of the optional necktie protector being folded over a necktie and also showing a required tie clip.

FIG. 6 is a plan view showing an EMT or other health care provider wearing the optional tie protector of FIG. 5 as a protective bib.

FIG. 7 is a plan view of the arm wrap and cover for a blood pressure (BP) cuff of this invention.

FIG. 8 is a perspective detail showing the wrap of FIG. 7 being placed on the patient arm.

FIG. 9 is the next step which wraps and secures the BP cuff over the designated region of the wrap of FIG. 8.

FIG. 10 shows the last step of folding down the protective cover portion of the wrap over the BP cuff.

FIG. 11 shows an alternative embodiment of barriers/sheathes mounted on a substrate wherein the full stethoscope cover has been replaced by a head-only cover.

FIG. 12 shows a perspective detail of the stethoscope head cover of FIG. 11 in use.

### DETAILED DESCRIPTION OF THE INVENTION

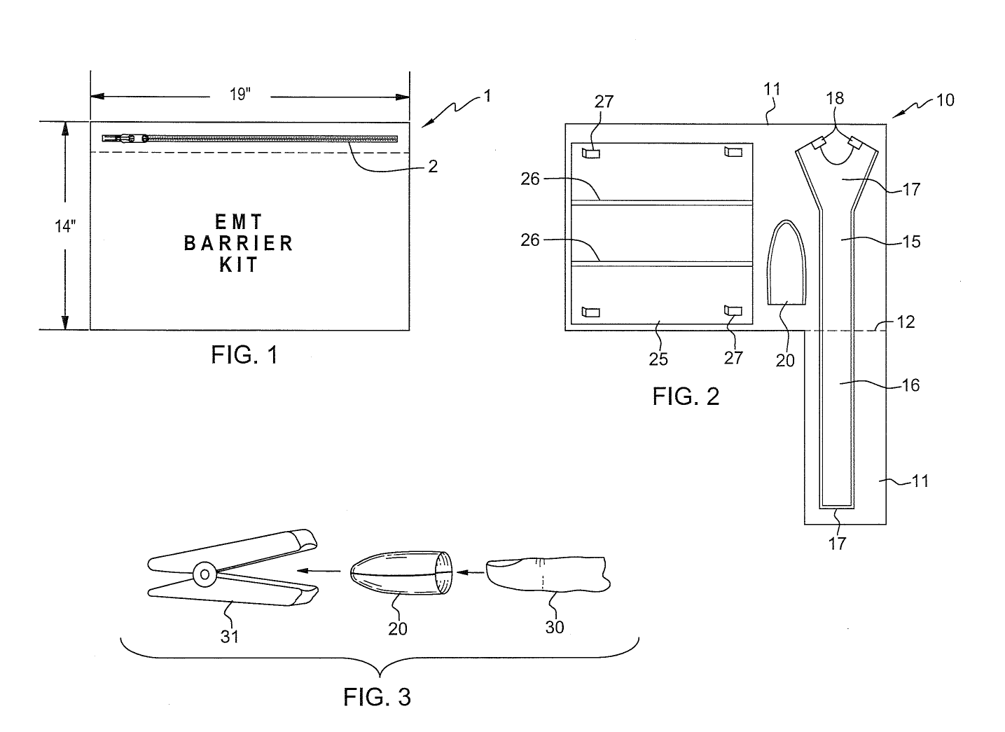

Since space is at a premium within an ambulance, It has been attempted to make this barrier/sheath kit as compact as possible while maintaining ease of use. It is possible to reconfigure the kit to be possibly more conformable to spaces available in the ambulance without deviating from the scope of the invention. For this description, envelope 1 of FIG. 1 of the dimensions shown (and ⅛″ thick) contains all of the items within a barrier paper envelope made of material similar to that used to package sterile adhesive bandages and pads. Item 2 is a zip opening that maintains a sterile seal (or a cutting string).

FIG. 2 shows a family of barriers/sheaths formed of the same material (eg. 60 to 100 GA. vinyl or polyethylene plastic film) mounted on cardboard substrate 11. Although other manufacturing techniques can be used, an efficient semi-automated method involves treating one side of cardboard 11 surface with a very low tack adhesive, laying flat a single surface of plastic film with a second film surface on those areas requiring two layers. Then, the edges of the two layer parts are bonded by heat sealing where needed. A following step involves die cutting of the plastic film using a steel rule die of appropriate shapes to cut the individual barriers or sheaths keeping them attached to the cardboard to be separated by the EMT when needed.

Item 20 is a sheath for use over a patient finger isolating contact with a pulse oximeter or pulse oximeter probe. Note that the edge is heat sealed (double lines shown) except for the end. FIG. 3 shows a patient finger 30 poised to enter sheath 20 and then further insertion into a pulse oximeter 31. A paper entitled “The effect of a disposable probe cover on pulse oximetry” by Cheung, Hardman, and Whiteside was published in the Apr. 30, 2002 issue of Anaesth Intensive Care. The limited study indicates that a polyethylene cover may introduce a small error in pulse oximetry reading, but “This error is unlikely to be of clinical significance”. Since this is the only usage of barrier material in this “family” that could impact actual readings or metrics, it should be the one determining the actual barrier material selected based on further testing.

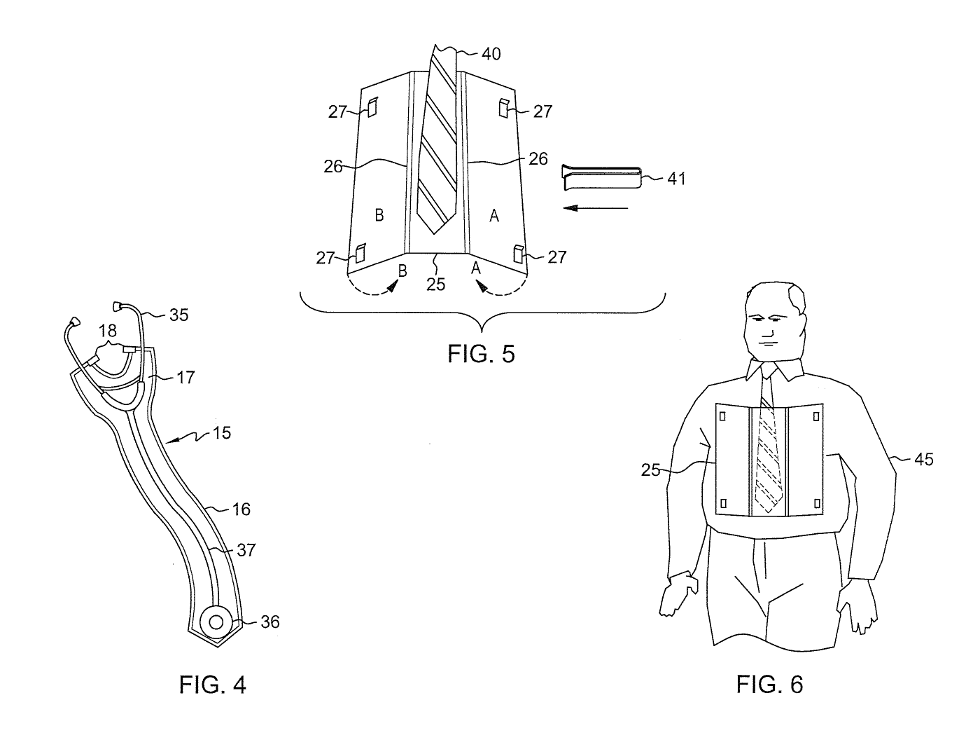

Item 15 with closed distal end 17 and open top Y section 17 is for covering a stethoscope. Adhesive tabs 18 attach sheath 15 around the tubing forming the ear pipes of stethoscope 35 in FIG. 4 which shows sheath 15 in use. Long section 16 houses the sound hose 37 and is wide enough to accommodate stethoscope head 36 which obviates the need for a separate head or diaphragm cover as in the prior art. Note that cardboard substrate 11 is folded up at site 12 to permitting fitting in envelope 1.

Item 25 is a single layer sheet with two formed fold lines 26 (heated to a low is temperature). This is the optional necktie sheath which can also double as a protective bib to limit EMT/patient contact. It has four adhesive patches 27 preferably formed of cut lengths of double sided tape such as Scotch Brand type 9415PC with high/low tack film with poly coated liner. This would have the high tack adhesive side against the plastic film with the low tack adhesive under the easily removable liner. The necktie sheath use is illustrated in FIG. 5 where release liner is removed from the two patches 27 on panel B but not removed from panel A. Then sheath 25 is positioned as shown behind the lower portion of necktie 40 and panel A is folded inwards against the necktie. Then panel B is folded inwards over panel A with patches 27 attaching B to A with low tack (for easy removal). A tie clip such as 41 is required to attach the assembly to a shirt to keep it from touching a patient when an EMT or other health care provider leans over (main mode of cross contamination for the unprotected necktie!). As an alternative, panel 25 can be used open as a barrier in the form of a bib attached to the front shirt portion of EMT 45 as shown in FIG. 6. Here, the release liners from all four patches 27 are removed to provide 4 points of shirt attachment. Note that if a tie is worn, this also prevents cross contamination when used in this open mode.

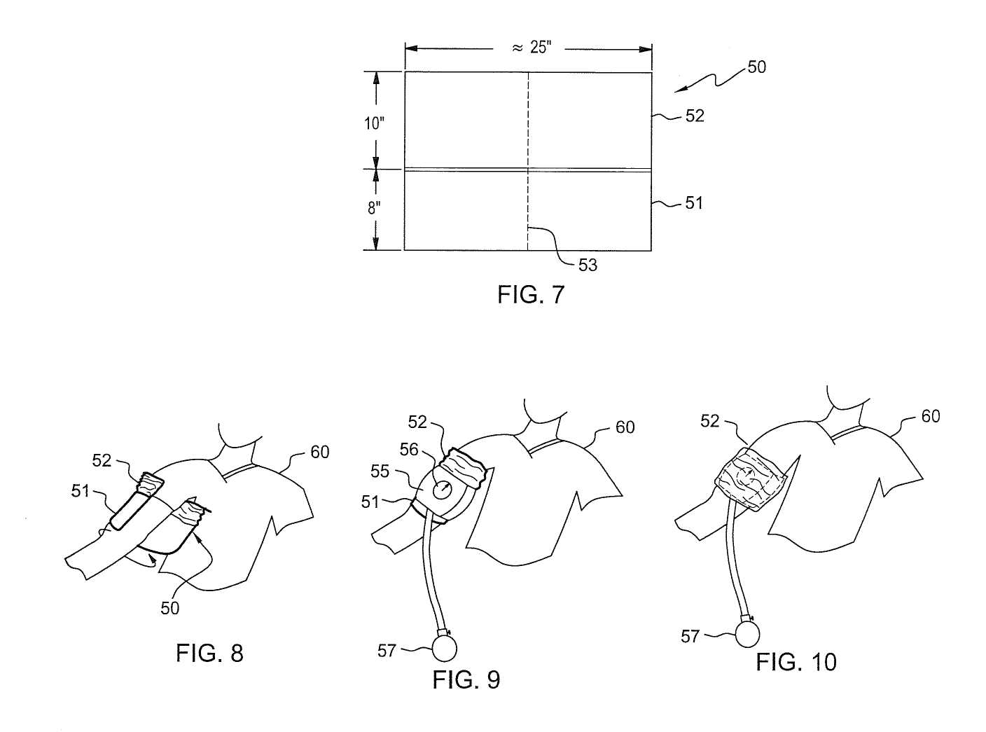

FIGS. 7-10 relate to a separate barrier sheet 50 of different material that is also enclosed in sterile envelope 1. This is a rectangular sheet of approximate dimensions as shown in FIG. 7. It consists of two bands of blown stretch wrap (linear low density polyethylene-LLDPE). The 10″ (25 cm) band is made of 60 GA or thinner film while the 8″ (20 cm) to which it is linearly bonded is of 120 GA stretch wrap. Note that sheet 50 is folded at line 53 over a separator sheet (not shown) of paper with a non-stick coating to facilitate fitting in sterile envelope 1. Usage is a simple fast three step process. Sheet 50 is wrapped around the upper arm of a patient 60 as in FIG. 8 with thinner material band 52 pushed and distorted toward the armpit. Sheet 50 should self-adhere with low tack to stay in place with section 51 providing a smooth base surface for installation of a typical blood pressure (BP) cuff 55 with pressure gage 56 as in FIG. 9. The last step in FIG. 10 is to pull down and forward on layer 52 over cuff 55 to protect against splatter of bodily fluids. Note that as per the cleaning instructions, if the cuff Velcro gets contaminated the BP cuff must be discarded as “red” waste. The entire BP cuff 55 should be protected except for pressurizing bulb 57 and its flexible line which can be easily cleaned by wiping. Barrier sheet 50 isolates the inner surface of BP cuff 55 from touching patient skin. Note that layer 52 is transparent and pressure gage 56 may be read adequately through it, but it can also be raised up is while actual reading is taken.

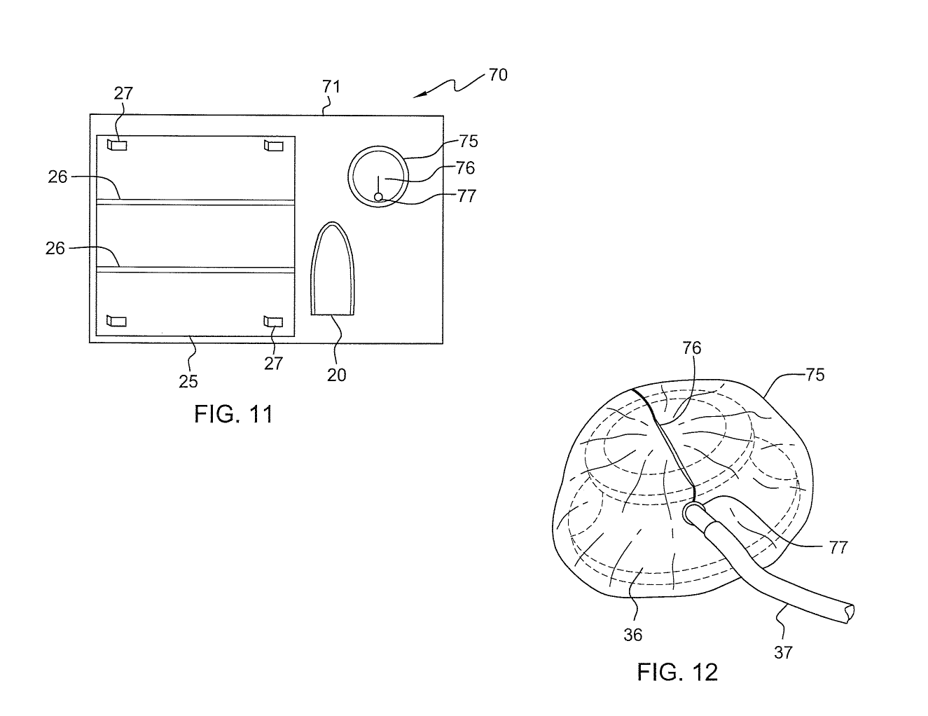

FIGS. 11 and 12 relate to a substitution of a stethoscope head cover 75 for the full stethoscope cover 15 shown in FIG. 2. Head cover 75 is circular in shape (approximately 2.75″ in diameter or 70 mm when flat). It has a solid bottom layer which is heat sealed around the edge to a top layer with access slit 76 and hose connection hole 77 near the edge. This is shown in FIG. 11 as part of family 70; note also that cardboard substrate 71 no longer requires the folded extension of substrate 11 shown in FIG. 2.

FIG. 12 shows stethoscope head 36 installed through slit 76 inside cover 75. Note that hose 37 fits through hole 77 at the end of slit 76. Although head cover 75 could be a rectangular or square shape, a round shape was selected to offer better conformability to the round head 36. In the foregoing description, certain terms and visual depictions are used to illustrate the preferred embodiment. However, no unnecessary limitations are to be construed by the terms used or illustrations depicted, beyond what is shown in the prior art, since the terms and illustrations are exemplary only, and are not meant to limit the scope of the present invention.

It is further known that other modifications may be made to the present invention, without departing the scope of the invention, as noted in the appended Claims.

I claim:

1. A barrier kit for EMT monitoring equipment comprising:
a sterile envelope of disposable barrier material;
said envelope containing a board of disposable sterile material on which are mounted a family of sanitary barriers and sheaths;
said board having a releasable adhesive on one surface thereof on which said barriers and sheaths are releasably mounted; and
said family of barriers and sheaths comprising a finger sheath adapted for use over a patient finger for isolating contact with a pulse oximeter or pulse oximeter probe, a barrier cover for a stethoscope, a single layer sheet with fold lines adapted to enclose a tie or chest region of an emergency medical personnel person, and a barrier sheet adapted for wrapping around an upper arm of a patient to provide a smooth base surface for a blood pressure cuff; and
said barriers and sheaths being made from materials resistant to migration of microbes therethrough.

2. The barrier kit of claim 1 in which said barrier cover for a stethoscope comprises a long section for enclosing a sound hose of said stethoscope, a stethoscope head at a proximal end thereof, and tubing forming ear pipes at an open Y-shaped section at a distal end thereof, long section being foldable for mounting on said board so that said board fits within said envelope.

3. The barrier kit of claim 2 in which said Y-shaped section is provided with a pair of adhesive tabs for attaching said Y-shaped section around said tubing.

4. The barrier kit of claim 1 in which said single layer sheet has a pair of spaced, parallel fold lines for enclosing said tie, said single layer barrier sheet being retained on said board by a plurality of releasable adhesive patches.

5. The barrier kit of claim 4 in which said kit includes a tie pin for securing the single layer sheet enclosing a tie to a shirt of an emergency medical personnel person.

6. The barrier kit of claim 4 in which said single layer sheet is adaptable for use as a bib when attached to a shirt of the EMT attending the patient.

7. The barrier kit of claim 1 in which said barrier sheet is comprised of narrower and wider bands of stretch wrap, said wider band providing a smooth base surface for installation of a blood pressure cuff, said narrower band being thinner, said wider band having a low tack surface on a side facing the arm of the patient.

8. The barrier kit of claim 1 wherein said barrier cover for said stethoscope comprises a head cover with a flat bottom surface for mounting on said board, said head cover having an access slit on a top surface thereof, said head cover being round to accommodate and enclose the head portion of said stethoscope.

9. A method for using disposable sanitary covers for patient monitoring equipment in emergency rescue and other medical environments comprising the steps of:
providing sterile envelopes of disposable barrier material in an EMT emergency vehicle;
inserting into each said envelope a board of disposable sterile material on which are mounted a family of sanitary barriers and sheaths, said board having a releasable adhesive on one surface thereof on which said barriers and sheaths are releasably mounted, said family of barriers and sheaths comprising a sheath adapted for use over a patient finger for isolating contact with a pulse oximeter or pulse oximeter probe, a barrier cover for a stethoscope, and a single layer sheet with fold lines adapted to enclose a tie or chest region of an emergency medical personnel person, said barriers and sheaths being made from materials resistant to migration of microbes therethrough;
separately enclosed in said sterile envelope, a barrier sheet adapted for wrapping around an upper arm of a patient to provide a smooth base surface for a blood pressure cuff; said sheet being made from material resistant to migration of microbes therethrough;
carrying said envelopes containing said families of sanitary barriers and sheaths in said emergency vehicle carrying medical technicians when travelling to a site having a medical emergency;
in an emergency situation dealing with a victim patient, opening one of said envelopes and withdrawing the board within with said family mounted thereon;
removing said finger sheath from said board and sliding said finger sheath onto a distal end of a finger of said patient in preparation for measuring oxygen levels in blood of the patient;
removing said barrier cover and enclosing said stethoscope in said barrier cover prior to deploying said stethoscope;
using said single layer sheet to enclose a tie of an emergency medical personnel person if the emergency medical personnel person is wearing a tie or deploying said single layer sheet as a bib on a medical technician;
wrapping said barrier sheet around the upper arm of said patient with said narrower band being closest to the arm pit of the patient, followed by placing said blood pressure cuff around the wider band of said barrier sheet and then pulling said narrower band down covering said blood pressure cuff; and
said barriers and sheaths being made from materials resistant to migration of microbes therethrough whereby cross contamination between successive patients is reduced.

10. A barrier kit for EMT monitoring equipment comprising:
a sterile envelope of disposable barrier material;
said envelope containing a board of disposable sterile material on which are mounted a family of sanitary barriers and sheaths;
said board having a releasable adhesive on one surface thereof on which said barriers and sheaths are releasably mounted; and
said family of barriers and sheaths comprising a finger sheath adapted for use over a patient finger for isolating contact with a pulse oximeter or pulse oximeter probe, a barrier cover for a stethoscope, and a barrier sheet adapted for wrapping around an upper arm of a patient to provide a smooth base surface for a blood pressure cuff; and
said barriers and sheaths being made from materials resistant to migration of microbes therethrough.

11. The barrier kit of claim 10 further comprising a single layer sheet with fold lines adapted to enclose a tie or chest region of an emergency medical personnel person.

12. The barrier kit of claim 10 in which said barrier cover for a stethoscope comprises a long section for enclosing a sound hose of said stethoscope, a stethoscope head at a proximal end thereof, and tubing forming ear pipes at an open Y-shaped section at a distal end thereof, long section being foldable for mounting on said board so that said board fits within said envelope.

13. The barrier kit of claim 12 in which said Y-shaped section is provided with a pair of adhesive tabs for attaching said. Y-shaped section around said tubing.

14. The barrier kit of claim 11 in which said single layer sheet has a pair of spaced, parallel fold lines for enclosing said tie, said single layer barrier sheet being retained on said board by a plurality of releasable adhesive patches.

15. The barrier kit of claim 14 in which said kit includes a tie pin for securing the single layer sheet enclosing a tie to a shirt of an emergency medical personnel person.

16. The barrier kit of claim 11 in which said single layer sheet is adaptable for use as a bib when attached to a shirt of the EMT attending the patient.

17. The barrier kit of claim 10 in which said barrier sheet is comprised of narrower and wider bands of stretch wrap, said wider band providing a smooth base surface for installation of a blood pressure cuff, said narrower band being thinner, said wider band having a low tack surface on a side facing the arm of the patient.

18. The barrier kit of claim 10 wherein said barrier cover for said stethoscope comprises a head cover with a flat bottom surface for mounting on said board, said head cover having an access slit on a top surface thereof, said head cover being round to accommodate and enclose the head portion of said stethoscope.
